(19) United States
(12) Patent Application Publication
(10) Pub. No.: US 2009/0110720 A1
(43) Pub. Date: Apr. 30, 2009

(54) Use of compounds for the prevention of drug-induced cell toxicity

(76) Inventor: Anders Nykjaer, Risskov (DK)

Correspondence Address: BROWDY AND NEIMARK, P.L.L.C.;624 NINTH STREET, NW, SUITE 300, WASHINGTON, DC, 20001-5303, US

(21) Appl. No.: 11/664,789

(22) PCT Filed: Oct. 5, 2005
(86) PCT No.: PCT/DK05/00640

§ 371 Date: Apr. 6, 2007

(51) Int. Cl. A61K 9/127; A61K 31/7028

(52) U.S. Cl. 424/450; 514/23

(57) ABSTRACT

The present invention relates to the use of compounds for the manufacture of a medicament for the prophylaxis and/or treatment of induced cell toxicity, such as nephewrotoxicity and ototoxicity, in particular where the cell toxicity is induced by a medical treatment. In a preferred embodiment, the compounds have at least two nitrogen atoms, more preferably at least two amino groups. The compounds according to the invention are capable of docking binding of cell toxic compounds to the megalin receptor, and thereby inhibiting uptake of the cell toxic compounds into cells. The invention further relates to novel compounds for use in said treatment, as well as a method for reducing the cell toxicity of cell toxic compounds.

All patent and non-patent references cited in the present application are hereby incorporated by reference in their entirety.

### FIELD OF INVENTION

The present invention relates to the technical field of cell toxicity treatment and discloses compounds and combination medicaments for use in such treatment.

### BACKGROUND OF INVENTION

The invention relates to compounds for the prevention of organ damage, in particular organ damage of the kidneys and the inner ear induced by the administration of therapeutic agents.

Several classes of drugs in clinical use are toxic to tissues like the kidney and inner ear. Prominent drugs in this category are cisplatin, ifosfomide, cyclosporine, amphotericin B, valproate, polymyxin B and therapeutic antibodies. However, of particular importance are aminoglycosides which are among the most important antibiotics for the treatment of severe bacterial infections. They are the preferred agents against a number of Gram-negative bacteria. At present the market share of aminoglycosides in the field of anti-infectious agents is rapidly increasing. Above all, this is due to a general increase in the occurrence of pathological strains resistant to other classes of antibiotics.

The main obstacle in the clinical use of the above drugs is their severe oto- and nephrotoxic (ear and kidney) side effects, which may lead to complete loss of hearing and to renal failure in the long term. The use of these drugs is thus not only associated with a high risk but also entails high costs for drug monitoring and diagnosis. Their use is therefore restricted to incidences of the most severe infections in the industrial countries. In the developing countries, where aminoglycosides are used more frequently because of their low production costs, aminoglycosides account for 70% of all cases of acquired deafness.

The underlying mechanisms causing toxicity are not understood. So far it is known that the drugs bind to the surface of cells in the kidneys and the inner ear and are taken up into the cells through unknown mechanisms. As the drugs are poorly degradable in the cells, they accumulate intracellularly leading to the destruction of cell structures and thus to renal damage and hearing loss. Various surface structures or receptors have been held responsible for the binding and uptake of the antibiotics.

Moestrup et al. suggested that megalin, a surface receptor of the kidneys, is responsible for the uptake of antibiotics (Moestrup et al., J. Clin. Invest. 96, 1404-1413, 1995). Megalin is a 600 kDa endocytosis receptor of the low-density lipoprotein (LDL) receptor gene family. Megalin is a multifunctional clearance receptor that binds and internalises a number of macromolecules. The sequence for megalin is shown as: cDNA: U33837; gene: NT_{—}002176.

Another receptor believed to be involved in antibiotic interaction is cubilin. Cubilin is a 460 kDa membrane-associated protein colocalizing with megalin, that may facilitate the endocytic process by sequestering the antibiotic/therapeutic agent on the cellular surface before megalin-mediated internalization of the cubilin-bound ligand. In other words, the therapeutic agent may bind to cubilin as well as directly to megalin. Cubilin, however, appears not to be able to mediate endocytosis on its own but physically associates with megalin and internalizes in a complex with this receptor. The sequence for cubilin is shown as: cDNA: XM_{—}011904; gene: NT_{—}008682 (Homo sapiens chromosome 10 working draft sequence segment).

Several strategies for preventing toxic side effects of aminoglycosides have been developed. Examples are the development of novel aminoglycosides having fewer side effects (see amikacin, a semi-synthetic derivative of kanamycin (Begg, E. J. & Barclay, M. L. Br. J. Clin. Pharmac. 39, 597-603, 1995)), and the simultaneous administration of aminoglycosides with other compounds, such as neurotrophin-3 (Ernfors, P., Duan, M. L., ElShamy, W. M. & Canlon, B. Nat. Med. 2, 463-467 (1996)), nitrendipine (Lee, S. M., Pattison, M. E. & Michael, U. F. J. Cardiovasc. Pharmacol. 9, S65-S69 (1997)), Pyrola rotundifolia (Xuan, W. & Dong, M. Ann. Otol. Rhinol. Laryngol. 104, 374-380 (1995)), antioxidants (Schacht, J. Head and Neck Surgery 118, 674-677 (1998)), or, for ototoxicity, guanidine-analogs (WO 99/02145).

In Jones et al. (Jones, M. M., Basinger, M. A., and Holscher, M. A.; Fundamental and applied toxicology, 18, 181-188 (1992)) the control of nephrotoxicity of cisplatin by clinically using sulphur-containing compounds is described. Jones et al. disclose how sulphur-containing compounds bind to hydrolytic products derived from the platin part of cisplatin and thereby reduce the nephrotoxic side effect of cisplatin. The article does not disclose cell toxicity reducing compounds or medicaments capable of binding to a receptor cubilin and/or a receptor megalin and/or a co-receptor of megalin and cubilin.

### SUMMARY OF INVENTION

The present invention presents compounds having an improved cell toxicity reducing effect when used alone or in combination with a therapeutic agent causing said cell toxicity. It is furthermore an object of the invention to provide compounds for this use that themselves do not induce unacceptable toxicity when used in medical treatment.

In a first main aspect, the invention relates to the use of a compound comprises a structure of the general formula (I)

R₇-R₁-X-R₈

wherein

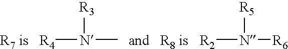

wherein
X is a bond or an aromatic, a carbocyclic, a heterocyclic or a heteroaromatic structure having 1-3 rings, 3-8 ring members in each and 0 to 3 heteroatoms, wherein each ring may be substituted at least once, wherein the substituents are selected from: O, OH, phenyl, halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, nitro, Oalkyl, Oacyl, aminoalkyl and aminodialkyl,
R₁ is a bond, or C1-10 alkyl, optionally substituted at least once, wherein the substituents are selected from: O, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, or nitro, wherein any of the carbons in said C1-10 alkyl is optionally replaced by oxygen, nitrogen, sulphur, or silicon,
R₂ is a bond or C1-10 alkyl, optionally substituted at least once, wherein the substituents are selected from: O, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, or nitro, wherein any of the carbons in said C1-10 alkyl is optionally replaced by oxygen, nitrogen, sulphur, or silicon,
R₃, R₄, R₅, and R₆ individually are selected from: a bond connecting to X of formula (I), hydrogen, OH, trifluoromethyl, cyano, amino, nitro, alkyl, alkenyl, alkynyl, phenyl, benzyl, halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl,
optionally further substituted one or more times with C, S, N, O, OH, phenyl, amine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, or nitro,
   wherein one or more of R₃, R₄, R₅, and R₆ is optionally linked to another substituent R₁-R₈ and/or to X, thereby forming a ring,
   wherein N′ and N″ are separated by at least 4 atoms,
   wherein N′ and N″ optionally have a further group attached thus forming a quaternary ammonium,
   with the proviso that the compound is not a diaminoalkyl, wherein both the alkyl group and the amino groups have no substitutions,
   and the proviso that the compound is not 3-methylamino-1-(4-methylpiperazino)-2-propanole, 4-piperazinoaniline, 2-[4-(2-aminoethyl)piperazin-1-yl]ethylamine, arginine, or 1-(2-pyrimidyl)-piperazine dihydrochloride
   or a pharmaceutically acceptable addition salt or hydrate of said compound,
   for the manufacture of a medicament for the prophylaxis and/or treatment of induced cell toxicity, in particular nephrotoxicity and/or ototoxicity, i.e. damage of the kidney and/or inner ear.

Similarly, the invention, in one aspect, relates to a method for the treatment and/or prophylactic treatment of induced cell toxicity, in particular nephrotoxicity and/or ototoxicity, comprising the administration of a compound of formula (I) to a person in need thereof.

When any variable occurs more than once in any constituent, each definition is independent.

In a further main aspect, the invention relates to a compound having the general formula of

(A_{q}-Y)ₚ

wherein
A is independently selected from formula (I) as defined herein, and wherein
Y is a spacer,
q is an integer of 1-100,
p is an integer of 1-100.

In another aspect of the invention, a combination medicament comprising a compound of the invention or any of the compounds for which a use is described herein and a therapeutic agent for simultaneous, separate or sequential use in cell toxicity therapy, said cell presenting a receptor megalin and/or a receptor cubilin and/or a co-receptor of megalin and cubilin is disclosed.

### DESCRIPTION OF DRAWINGS

FIG. 1: shows examples of compounds used in the invention

FIG. 2: inhibition of gentamicin binding to immobilized megalin by various compounds as assessed by surface plasmon resonance.

FIG. 3: inhibition of gentamicin binding to immobilized megalin by p-xylylene diamine as assessed by surface plasmon resonance.

FIG. 4: efficacy of p-xylylene diamine (RC043) in preventing renal uptake of gentamicin in the mouse kidney.

### DETAILED DESCRIPTION OF THE INVENTION

### Definitions

Alkyl group: the term “alkyl group” means a saturated linear or branched hydrocarbon group including, for example, methyl, ethyl, isopropyl, t-butyl, heptyl, dodecyl, octadecyl, amyl, 2-ethylhexyl, and the like. Preferred alkyls are lower alkyls, i.e. alkyls having 1 to 6 carbon atoms, such as 1, 2, 3, 4, 5 or 6 carbon atoms.

Alkenyl croup: the term “alkenyl” means a non-saturated linear or branched hydrocarbon group including, for example, methylene or ethylene.

Alkynyl group: the term “alkynyl” means a non-saturated linear or branched hydrocarbon group including, for example, ethynyl or propynyl.

Substituted lower alkyl means a lower alkyl having one to three substituents selected from the group consisting of hydroxyl, alkoxy, amino, amido, carboxyl, acyl, halogen, cyano, nitro and thiol.

Aryl represents a hydrocarbon comprising at least one aromatic ring, and may contain from 5 to 18, preferably from 6 to 14, more preferably from 6 to 10, and most preferably 6 carbon atoms. Typical aryl groups include phenyl, naphthyl, phenanthryl, anthracyl, indenyl, azulenyl, biphenylenyl, and fluorenyl groups. Particularly preferred aryl groups include phenyl, naphthyl and fluorenyl, with phenyl being most preferable.

Heterocyclyl means a monovalent saturated cyclic radical, consisting of one to two rings, of three to eight atoms per ring, incorporating one or two ring heteroatoms, chosen from N, O or S(O)₀₋₂, and which can optionally be substituted with one or two substituents selected from the group consisting of hydroxyl, oxo, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminofarbonyl, arylaminocarbonyl, alkylcarbonylamino, or arylcarbonylamino.

Heteroaryl means a monovalent aromatic cyclic radical having one to three rings, of four to eight atoms per ring, incorporating one or two heteroatoms (chosen from nitrogen, oxygen, or sulfur) within the ring which can optionally be substituted with one or two substituents selected from the group consisting of hydroxy, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonlamino and arylcarbonylamino.

Cycloalkyl means a monovalent saturated carbocyclic radical consisting of one or two rings, of three to eight carbons per ring, which can optionally be substituted with one or two substituents selected from the group consisting of hydroxy, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino and arylcarbonylamino.

Induced cell toxicity means a toxic response induced by a therapeutic agent in a cell being exposed to said therapeutic agent.

Cell toxicity: an agent is defined as toxic when it is directly capable of causing cell death.

Cell death: in the present context cell death is defined in various ways and covers a cell which has lost all its functions, a cell which has lost a special function, such as hormone synthesis, or a cell which has a reduced capability for further division.

Spacer in the present context refers to the atoms directly linking the monomers of formula (I), (II) or (III). The spacer may also directly link the compounds of formula (I), (II) or (III) to a therapeutic agent as described by the present combination medicament.

Form a ring means that the atoms mentioned are connected through a bond when the ring structure is formed. The term “ring” is used synonymously with the term “cyclic”.

Therapeutic agent is used synonymously with a medicament, unless otherwise stated.

Prophylaxis or prophylactic treatment is not intended to be limited to complete prevention of induced cell toxicity, but also includes incomplete reduction of such toxicity.

### ASPECTS OF THE INVENTION

The principle of the present invention is to reduce the side effects caused by therapeutic agents, in particular kidney and inner ear damage. The toxicity of the therapeutic agents is possibly due to the accumulation of the therapeutic agent in cells in the organs in question. Thus, the invention is focused on the inhibition of the accumulation in the cells of the therapeutic agent.

Accordingly, the present invention relates to the use of novel compounds capable of inhibiting the intracellular accumulation. This may for example be done by inhibiting the binding of the therapeutic agent to the receptor megalin by either blocking a sufficient amount of binding sites on the receptor megalin and/or blocking the therapeutic agent so that it maintains the normal therapeutic effect but is inhibited from binding to the receptor.

By the present invention, the novel use of compounds acting as antagonists for use in the manufacture of medicaments for the prophylaxis and/or treatment of induced cell toxicity is disclosed.

### Novel Use of Compounds

It is within the scope of the invention that the compounds of the invention may bind to any given receptor involved in induced cell toxicity in order to inhibit binding and optionally uptake of the therapeutic agent into the cell.

The compound is preferably either capable of binding to a sufficient number of binding sites on the receptor(s) and/or of binding to the receptor and sterically hindering the binding of the therapeutic agent.

In a main aspect, the invention relates to the use of a compound comprises a structure of the general formula (I)

R₇-R₁-X-R₈

wherein

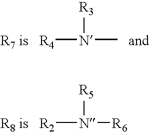

wherein
X is a bond or an aromatic, a carbocyclic, a heterocyclic or a heteroaromatic structure having 1-3 rings, 3-8 ring members in each and 0 to 3 heteroatoms, wherein each ring may be substituted at least once, wherein the substituents are selected from: O, OH, phenyl, halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, nitro, Oalkyl, Oacyl, aminoalkyl and aminodialkyl,
R₁ is a bond, or C1-10 alkyl, optionally substituted at least once, wherein the substituents are selected from: O, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, or nitro, wherein any of the carbons in said C1-10 alkyl is optionally replaced by oxygen, nitrogen, sulphur, or silicon,
R₂ is a bond or C1-10 alkyl, optionally substituted at least once, wherein the substituents are selected from: O, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, or nitro, wherein any of the carbons in said C1-10 alkyl is optionally replaced by oxygen, nitrogen, sulphur, or silicon,
R₃, R₄, R₅, and R₆ individually are selected from: a bond connecting to X of formula (I), hydrogen, OH, trifluoromethyl, cyano, amino, nitro, alkyl, alkenyl, alkynyl, phenyl, benzyl, halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl,
optionally further substituted one or more times with C, S, N, O, OH, phenyl, amine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, or nitro,
   wherein one or more of R₃, R₄, R₅, and R₆ is optionally linked to another substituent R₁-R₈ and/or to X, thereby forming a ring,
   wherein N′ and N″ are separated by at least 4 atoms,
   wherein N′ and N″ optionally have a further group attached thus forming a quaternary ammonium,
   with the proviso that the compound is not a diaminoalkyl, wherein both the alkyl group and the amino groups have no substitutions,
   and the proviso that the compound is not 3-methylamino-1-(4-methylpiperazino)-2-propanole, 4-piperazinoaniline, 2-[4-(2-aminoethyl)piperazin-1-yl]ethylamine, arginine, or 1-(2-pyrimidyl)-piperazine dihydrochloride
   or a pharmaceutically acceptable addition salt or hydrate of said compound of formula (I),
   for the manufacture of a medicament for the prophylaxis and/or treatment of induced cell toxicity, in particular nephrotoxicity and/or ototoxicity, i.e. damage of the kidney and/or inner ear.

When any variable occurs more than once in any constituent, each definition is independent.

When used herein, “separated by X atoms” refers to the shortest path from one atom to another atom in a molecule. For example, “wherein N′ and N″ are separated by 5 atoms” means that no path from N′ to N″ passes via fewer than 5 intermediate atoms.

In an important embodiment, a compound is used, wherein
X is an aromatic, a heteroaromatic or a 5 or 6 membered saturated ring containing 0-2 oxygen atoms, optionally substituted at least once, wherein the substituents are selected from: O, OH, phenyl, halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, nitro, Oalkyl, Oacyl, aminoalkyl and aminodialkyl,
   R₁ is C1-10 alkyl, optionally substituted at least once, wherein the substituents are selected from: O, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, and nitro, wherein any of the carbons in said C1-10 alkyl is optionally replaced by oxygen, nitrogen, sulphur, or silicon,
   R₂ is C1-10 alkyl, optionally substituted at least once, wherein the substituents are selected from: O, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, and nitro, wherein any of the carbons in said C1-10 alkyl is optionally replaced, by oxygen, nitrogen, sulphur, or silicon, and
   R₃, R₄, R₅, and R₆ individually are selected from: a bond connecting to X of formula (I), hydrogen, OH, trifluoromethyl, cyano, amino, nitro, alkyl, alkenyl, alkynyl, phenyl, benzyl, halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl,
optionally further substituted one or more times with C, S, N, O, OH, phenyl, amine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, or nitro.

In another aspect, the invention concerns the use of a compound comprising a structure of the general formula (I)

R₇-R₁-X-R₈

wherein

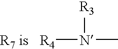

or a heterocyclic structure having 1-3 rings having at least one nitrogen and

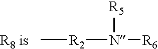

or a hydrogen,
wherein
N′ and N″ are nitrogen,
X is an aromatic, a carbocyclic, a heterocyclic or a heteroaromatic structure having 1-3 rings, 3-8 ring members in each and 0 to 3 heteroatoms, wherein each ring may be substituted at least once, wherein the substituents are selected from OH, phenyl, halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, and nitro,
R₁ is a bond, or C1-10 alkyl, optionally substituted at least once, wherein the substituents are selected from O, OH, phenyl, amine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, or nitro,
R₂ is a bond or C1-10 alkyl, optionally substituted at least once, wherein the substituents are selected from O, OH, phenyl, amine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, or nitro,
R₃, R₄, R₅, and R₆ individually is selected from: a bond connecting to X of formula (I), hydrogen, OH, trifluoromethyl, cyano, amino, nitro, alkyl, alkenyl, alkynyl, phenyl, benzyl, halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl,
optionally further substituted one or more times with C, S, N, O, OH, phenyl, amine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, or nitro,
   with the proviso that when R₁ or R₂ is a bond then none of R₃, R₄, R₅ and R₆ is a C substituted with both an amine and a nitrogen,
   and the proviso that when R₈ is a hydrogen, then X contains at least one nitrogen,
   or a pharmaceutically acceptable addition salt or hydrate thereof,
   for the manufacture of a medicament for the prophylaxis and/or treatment of induced cell toxicity.

Accordingly, in one main embodiment hereof, the compound comprises the general formula (II):

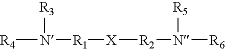

In another main embodiment hereof, the compound comprises the general formula (III):

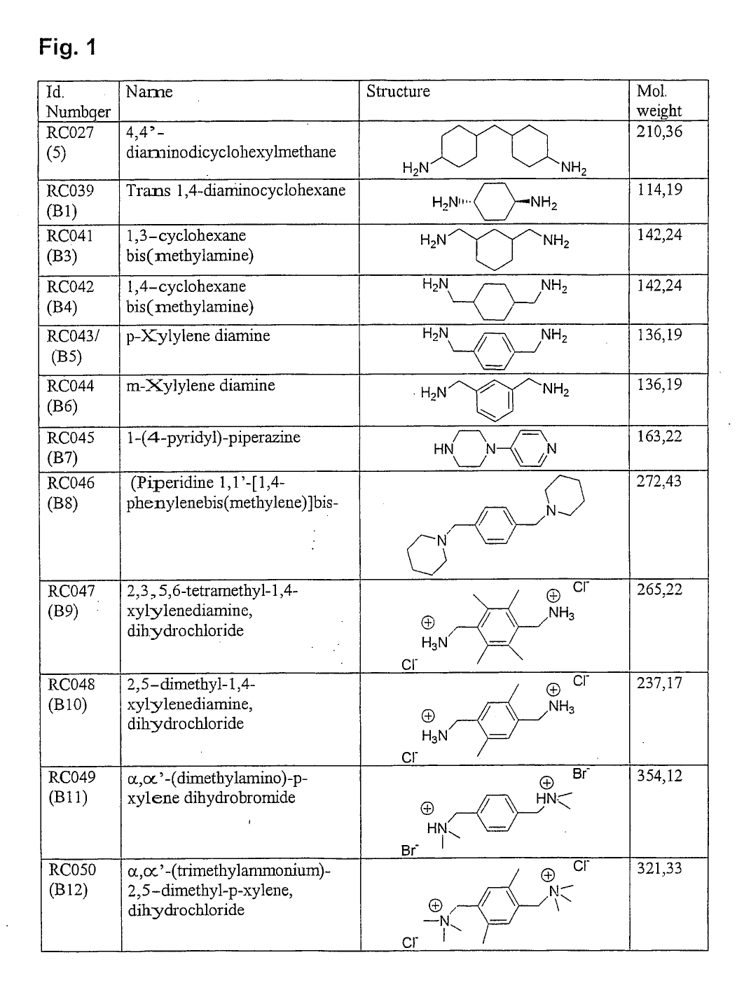

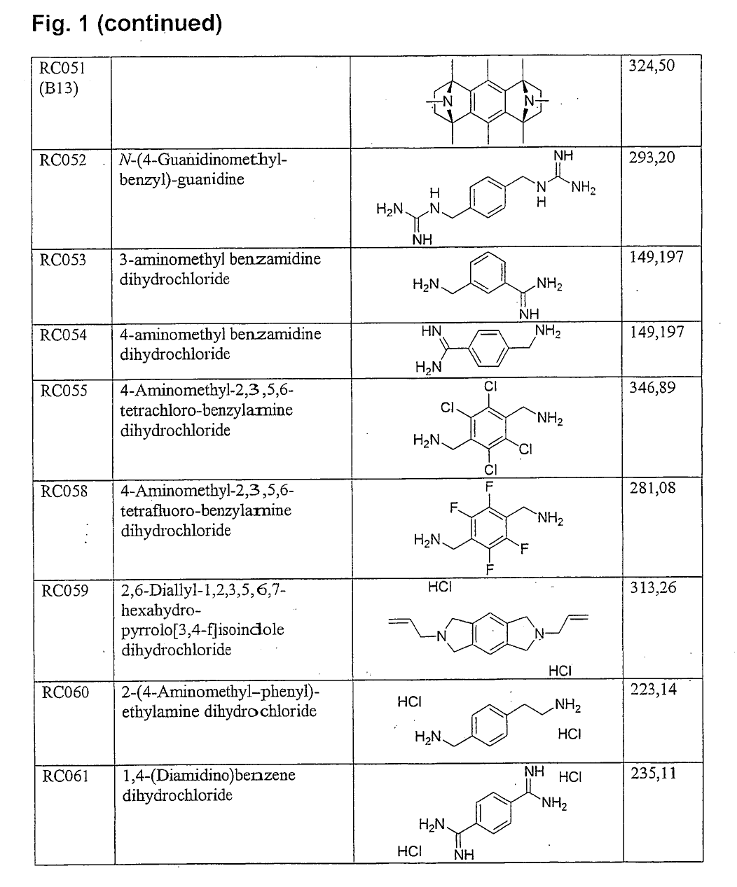

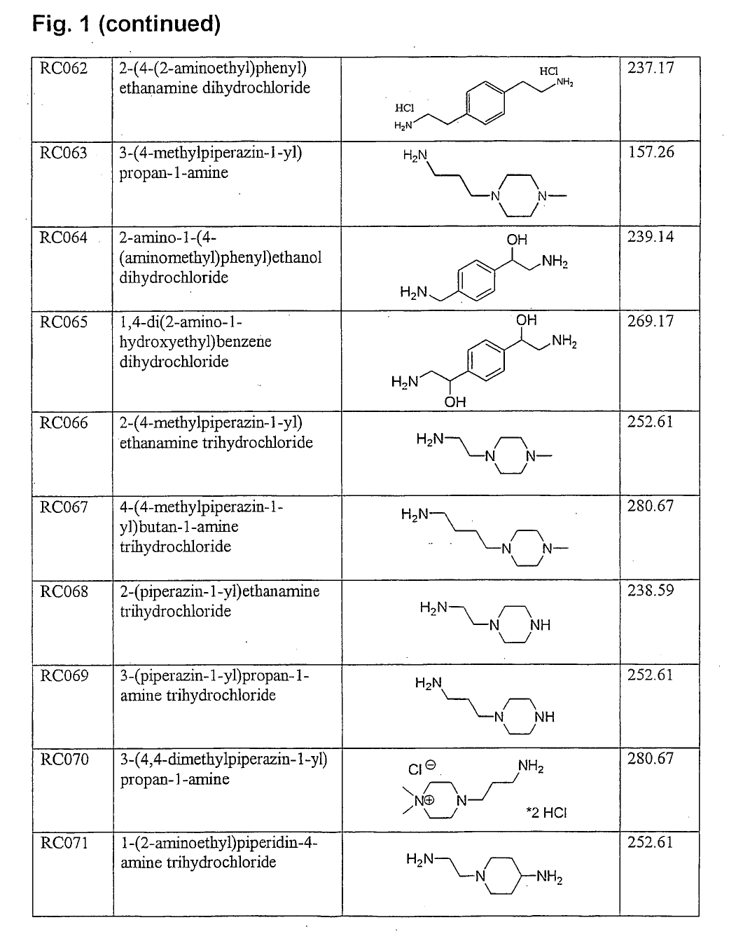

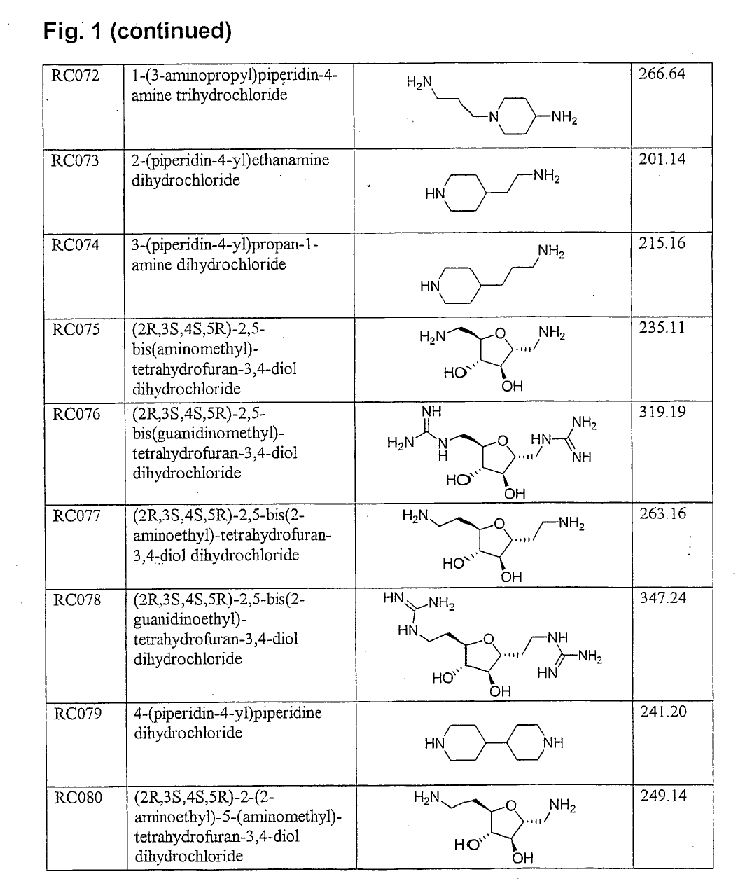

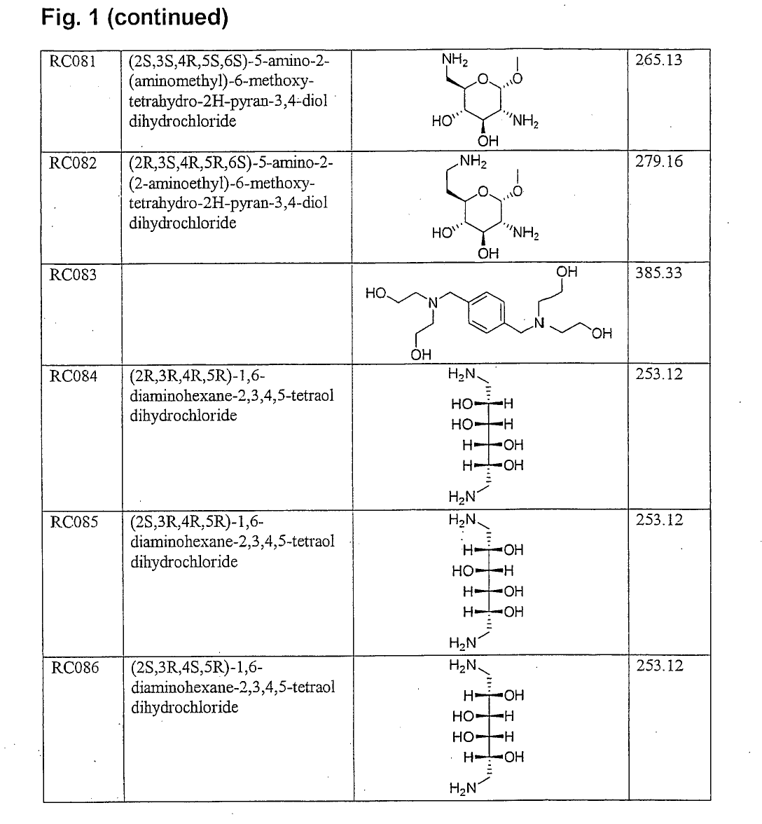

R₇-R₁-X—H

wherein R₇ is a heterocyclic structure having 1-3 rings having at least one nitrogen. A non-limiting example of a compound according to formula (III) is compound RC045 (B7) shown in FIG. 1.

Preferably, the cell in which toxicity is induced presents the receptor megalin and/or the receptor cubilin. More preferably, the cell is from the kidney and/or the inner ear.

### FURTHER EMBODIMENTS

### Preferred Embodiments of X

In preferred a embodiment of the use according to the invention, X is an aromatic, a carbocyclic, a heterocyclic or a heteroaromatic structure having 1-3 rings, 3-8 ring members and 0 to 3 heteroatoms in each ring, wherein each ring may be substituted at least once, wherein the substituents are selected from O, OH, phenyl, halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, nitro, Oalkyl, Oacyl, aminoalkyl and aminodialkyl.

In a further more preferred embodiment, wherein X is an aromatic, a carbocyclic, a heterocyclic or a heteroaromatic structure having 1-3 rings, 5-6 ring members and 1 or 2 heteroatoms in each ring, wherein each ring may be substituted at least once, wherein the substituents are selected from O, OH, phenyl, halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, nitro, Oalkyl, Oacyl, aminoalkyl and aminodialkyl.

In an even more preferred embodiment, X is an aromatic or a carbocyclic structure having 1-3 rings, 5-6 ring members and no heteroatoms in each ring, wherein each ring may be substituted at least once, wherein the substituents are selected from O, OH, phenyl, halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl(C₁₋₄)-alkyl, heterocyclyl-(C₁₋₄)-alkyl, cycloalkylalkyl, cycloalkyl, alkoxy, carboxy, trifluoromethyl, cyano, amino, nitro, Oalkyl, Oacyl, aminoalkyl and aminodialkyl.

In a further more preferred embodiment, X in the general formula (I), (II) or (III) given above is an aromatic, a carbocyclic, a heterocyclic or a heteroaromatic structure having 1-2 rings, 3-8 ring members in each and having 0 to 3 heteroatoms, wherein each ring optionally is substituted.

In another more preferred embodiment, X in the general formula (I), (II) or (III) given above is an aromatic, a carbocyclic, a heterocyclic or a heteroaromatic structure having 1 ring with 3-8 ring members and having 0 to 3 heteroatoms, said ring optionally being substituted.

In another even more preferred embodiment, X in the general formula (I), (II) or (III) given above is an aromatic, a carbocyclic, a heterocyclic or a heteroaromatic structure having 1 ring with 4-7 ring members and having 0 to 3 heteroatoms, said ring optionally being substituted.

In a yet more preferred embodiment, X in the general formula (I), (II) or (III) given above is an aromatic, a carbocyclic, a heterocyclic or a heteroaromatic structure having 1 ring with 5-6 ring members and having 0 to 3 heteroatoms, said ring optionally being substituted.

In a highly preferred embodiment, X in the general formula (I), (II) or (III) given above is an aromatic, a carbocyclic, a heterocyclic or a heteroaromatic structure having 1 ring with 5 ring members and having 0 to 1 heteroatoms, said ring optionally being substituted. X being preferably selected from the group consisting of cyclopentyl, furan, thiophene, pyrrole, imidazole, oxazole, and pyrrolidene, preferably the group consisting of furan and pyrrole.

In another highly preferred embodiment, X in the general formula (I), (II) or (III) given above is an aromatic, a carbocyclic, a heterocyclic or a heteroaromatic structure having 1 ring with 6 ring members and having 0 to 1 heteroatoms, said ring optionally being substituted. X being selected from the group consisting of pyridine, pyrimidine, cyclohexyl, and phenyl, preferably cyclohexyl and phenyl.

In another preferred embodiment, X in the general formula (III) given above is a heterocyclic structure, containing one or more nitrogen atoms, preferably one nitrogen atom, most preferably in the para-position relative to the bond with R₁.

In a further preferred embodiment, X is an aromatic, carbocyclic, a heterocyclic or a heteroaromatic structure having 1 ring with 6 ring members and having 0 to 1 heteroatoms, said ring optionally being substituted.

In another preferred embodiment, X is a dicyclohexylmethane.

In a further preferred embodiment, X comprises a heterocyclic ring comprising at least one oxygen atom.

In a highly preferred embodiment, compound comprises a structure of the formula V:

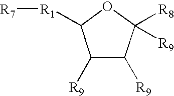

wherein each R₉, independently is H, OH, Oalkyl, Oacyl, halogen, alkyl, amino, aminoalkyl, aminodialkyl or aryl.

In another highly preferred embodiment, the compound comprises a structure of the formula VI:

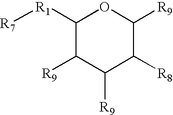

wherein each R₉, independently is H, OH, Oalkyl, Oacyl, halogen, alkyl, amino, aminoalkyl, aminodialkyl or aryl.

In a further highly preferred embodiment, the compound comprises a structure of the formula VII:

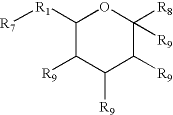

wherein each R₉, independently is H, OH, Oalkyl, Oacyl, halogen, alkyl, amino, aminoalkyl, aminodialkyl or aryl.

In a preferred embodiment of the compounds of V, VI or VII, at least one of the R₉ groups is H and at least one of the R₉ groups is OH. In another preferred embodiment of the compounds of V, VI or VII, R₇ comprises a guanidine group or moiety and/or R₈ comprises a guanidine group or moiety.

### Preferred Embodiments of R₁ and R₂

In one embodiment of the use of the invention, R₁ is a bond, or C1-10 alkyl, optionally substituted at least once, wherein the substituents are selected from O, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, and nitro.

In a preferred embodiment, R₁ is a bond, or C₁₋₄ alkyl, optionally substituted at least once, wherein the substituents are selected from O, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, and nitro.

In a further preferred embodiment, R₁ is a bond, or C₁₋₄ alkyl, optionally substituted at least once but at most four times, wherein the substituents are selected from O, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, and nitro.

In a more preferred embodiment, R₁ is a bond, or C₁₋₂ alkyl, optionally substituted at least once, wherein the substituents are selected from O, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, and nitro.

In an even more preferred embodiment, R₁ is a C1 or C2 alkyl, said alkyl being optionally substituted at least once, wherein the substituent(s) is selected from O, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, and nitro.

In a highly preferred embodiment, R₁ is a C1 or C2 alkyl, substituted at least once with an imine group or substituted at least once with an OH group.

In another highly preferred embodiment, wherein R₁ is C1 or a bond.

In a further preferred embodiment, the R₁ group, including any substituents (if present), consists in total of less than 25 atoms other than H, such as less than 10 atoms other than H, e.g. less than 8, such as less than 5, e.g. less than 4 atoms other than H, such as less than 3 atoms other than H.

In one embodiment of the use of the invention, R₂ is a bond or C1-10 alkyl, optionally substituted at least once, wherein the substituents are selected from O, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, and nitro.

In a preferred embodiment, R₂ is a bond or C1-4 alkyl, optionally substituted at least once, wherein the substituents are selected from O, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, and nitro.

In another preferred embodiment, R₂ is a bond or C1-4 alkyl, optionally substituted at least once but at most four times, wherein the substituents are selected from O, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, and nitro.

In a more preferred embodiment, R₂ is a bond or C1-2 alkyl, optionally substituted at least once, wherein the substituents are selected from O, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, and nitro.

In an even more preferred embodiment, R₂ is a C1 or C2 alkyl, said alkyl being optionally substituted at least once, wherein the substituents are selected from O, OH, phenyl, amine (NH₂), imine (NH), halogen, alkyl, alkenyl or alkynyl, substituted lower alkyl, substituted lower alkenyl or alkynyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, trifluoromethyl, cyano, amino, and nitro.

In a highly preferred embodiment, R₂ is a C1 or C2 alkyl, substituted at least once with an imine group or substituted at least once with an OH group.

In another highly preferred embodiment, R₂ is C1 or a bond.

In a further preferred embodiment, the R₂ group, including any substituents (if present), consists in total of less than 25 atoms other than H, such as less than 10 atoms other than H, e.g. less than 8, such as less than 5, e.g. less than 4 atoms other than H, such as less than 3 atoms other than H.

In a further highly preferred embodiment, R₁ is a bond and R₂ is a C₁₋₂ alkyl.

In further preferred embodiments of the use according to the invention, R₁ in the general formula (I), (II) or (III) given above is a bond or C1-4 alkyl, optionally mono- or disubstituted with a substituent selected from a lower alkyl and a cycloalkyl, preferably R₁ is a bond or C1-2 alkyl, optionally mono- or disubstituted with a substituent selected from a lower alkyl and a cycloalkyl.

Furthermore, in preferred embodiments of the use according to the invention, R₂ in the general formula (I), (II) or (I) given above is a bond or a C1-4 alkyl, optionally mono- or disubstituted with a substituent selected from a lower alkyl and a cycloalkyl, preferably R₂ is a bond or C1-2 alkyl, optionally mono- or disubstituted with a substituent selected from a lower alkyl and a cycloalkyl.

In a more preferred embodiment, R₁ in the general formula (I), (II) or (III) given above is a C1-4 alkyl or C1-4 alkenyl, optionally mono- or disubstituted with a substituent selected from a lower alkyl and a cycloalkyl, preferably R₂ is a C1-2 alkyl, optionally mono- or disubstituted with a substituent selected from a lower alkyl and a cycloalkyl.

In another more preferred embodiment, R₂ in the general formula (I), (II) or (III) given above is a C1-4 alkyl or C1-4 alkenyl, optionally mono- or disubstituted with a substituent selected from a lower alkyl and a cycloalkyl, preferably R₂ is a C1-2 alkyl, optionally mono- or disubstituted with a substituent selected from a lower alkyl and a cycloalkyl.

In another more preferred embodiment, R₁ in the general formula (I), (II) or (III) given above is methyl.

In another more preferred embodiment, R₂ in the general formula (I), (II) or (III) given above is methyl.

In another more preferred embodiment, R₁ in the general formula (I), (II) or (III) given above is a bond and R₂ in the general formulae given above is a C1-2 alkyl.

In another preferred embodiment, R₁ and R₂ in the general formula (I) or (II) are both a bond.

In a more preferred embodiment, R₁ in the general formula (III) given above is a bond.

### Preferred Embodiments of R₃, R₄, R₅, R₆, R₇ and R₈

In a preferred embodiment, R₃, R₄, R₅, and R₆ individually are selected from: hydrogen, OH, carboxy, halogen, trifluoromethyl, cyano, amino, nitro, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl,
optionally further substituted one or more times with C, S, N, O, OH, phenyl, amine (NH), halogen, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, halogen, trifluoromethyl, cyano, amino, or nitro,

In a preferred embodiment, at least one of R₃, R₄, R₅ and R₆ in the general formula (I), (II) or (III) given above is hydrogen.

In a more preferred embodiment, at least two of R₃, R₄, R₅ and R₆ in the general formula (I), (II) or (III) given above is hydrogen.

In an even more preferred embodiment, R₃, R₄, R₅ and R₆ are all hydrogen.

In a further preferred embodiment, at least one of R₃, R₄, R₅ and R₆ is an hydroxyalkyl, such as hydroxyethyl.

In another preferred embodiment, R₃ is linked to R₄, thereby forming a ring of 5-6 members and/or R₅ is linked to R₆, thereby forming a ring of 5-6 members.

In an even further preferred embodiment, R₇ is a guanidine group or moiety or comprises a guanidine group or moiety and/or R₈ is a guanidine group moiety or comprises a guanidine group or moiety.

In a yet further preferred embodiment, R₃ and/or R₅ are linked to X, thereby forming one or more rings. Preferably, at least one of said rings has 5 members.

In another preferred embodiment, R₃ is linked to X and to R₁, thereby generating a ring of 6 members, further comprising an atomic bridge. An example of such a compound is RC051.

In an even further preferred embodiment, R₄ is linked to X and to R₂, thereby generating a ring of 6 members, further comprising an atomic bridge. An example of such a compound is RC051.

In a further preferred embodiment, the R₃ group, including any substituents (if present), consists in total of less than 25 atoms other than H, such as less than 10 atoms other than H, e.g. less than 8, such as less than 5, e.g. less than 4 atoms other than H, such as less than 3 atoms other than H.

In a further preferred embodiment, the R₄ group, including any substituents (if present), consists in total of less than 25 atoms other than H, such as less than 10 atoms other than H, e.g. less than 8, such as less than 5, e.g. less than 4 atoms other than H, such as less than 3 atoms other than H.

In a further preferred embodiment, the R₅ group, including any substituents (if present), consists in total of less than 25 atoms other than H, such as less than 10 atoms other than H, e.g. less than 8, such as less than 5, e.g. less than 4 atoms other than H, such as less than 3 atoms other than H.

In a further preferred embodiment, the R₆ group, including any substituents (if present), consists in total of less than 25 atoms other than H, such as less than 10 atoms other than H, e.g. less than 8, such as less than 5, e.g. less than 4 atoms other than H, such as less than 3 atoms other than H.

In a further preferred embodiment, the R₇ group, including any substituents (if present), consists in total of less than 25 atoms other than H, such as less than 10 atoms other than H, e.g. less than 8, such as less than 5, e.g. less than 4 atoms other than H, such as less than 3 atoms other than H.

In a further preferred embodiment, the R₈ group, including any substituents (if present), consists in total of less than 25 atoms other than H, such as less than 10 atoms other than H, e.g. less than 8, such as less than 5, e.g. less than 4 atoms other than H, such as less than 3 atoms other than H.

### Further Piperidine and Piperazine Derivatives

In one embodiment, X, R₂, N″ and R₅ together form a ring Y, said ring Y being a piperazine or piperidine ring, thereby forming the general formula VIII:

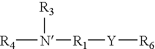

wherein R₁, R₃, R₄ and R₆ are as defined in any one of the embodiments mentioned above.

In a more preferred embodiment R₁ herein is an unsubstituted C2, C3 or C4 alkyl.

In a different more preferred embodiment, R₁ herein is a substituted C2, C3 or C4 alkyl.

In further preferred embodiments, R₃ is H and/or R₄ is H.

In a yet different preferred embodiment, R₁, R₃ and N′ together form piperazine or piperidine ring.

In any of the above embodiments, R₆ is preferably H or CH₃ or NH₂.

Further Embodiments wherein X is a Bond

In one embodiment, X is a bond in the compound of formula (I) or (II). More preferably, X is a bond and none of R₃, R₄, R₅, and R₆ is linked to another substituent R₁-R₈.

Even more preferably, R₁ is a bond and R₂ is C1-4 alkyl substituted at least once with an OH group and/or R₂ is a C4 alkyl substituted two, three or four times with an OH group, preferably on different C atoms.

Furthermore, one, two or three or all of R₃, R₄, R₅, and R₆ are preferably hydrogen.

### Provisos

In some preferred embodiments of the invention, the compound does not comprise a substituted or unsubstituted piperazine structure.

In other preferred embodiments of the invention, the compound does not comprise a substituted or unsubstituted piperidine structure.

In yet other preferred embodiments of the invention, the compound does not comprise a ring structure having 3 nitrogen atoms.

In a preferred embodiment of the use of the invention, in particular use in the prophylaxis of ototoxicity, the compound does not contain a guanidine moiety or derivative thereof, i.e. the following moiety:

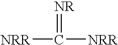

wherein R can be any group, atom or a bond.

In another more preferred embodiment, in particular use in the prophylaxis of ototoxicity the compound does not contain the moiety:

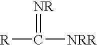

wherein R can be any group, atom or a bond.

In yet a further embodiment, the compound does not contain an adenosine or adenosyl moiety.

In an even further embodiment, the compound is not biotin when the agent inducing cell toxicity is cisplatin

In an even further embodiment, the compound is not 2,4-diamino-6-hydroxypyrimidine or 2-phthalimido acetamide.

In an even further preferred embodiment, the compound is not polymyxin, RAP (receptor-associated protein), poly-L-lysine, aprotinin or a fragment of any of these.

In an even further preferred embodiment, the compound is not any of: diaminomethane, 1,2-diaminoethane, 1,3-diaminopropane, 1,4-diaminobutane, 1,5-diaminopentane, 1,6-diaminohexane, 1,7-diaminoheptane, 1,8-diaminooctane, 3-methylamino-1-(4-methylpiperazino)-2-propanole, 4-piperazinoaniline, 1-(3-chlorophenyl)piperazine diHCl (m-CPP), piperazin-2-onel, 2-[4-(2-aminoethyl)piperazin-1-yl]ethylamine, piperazine anhydrous, 2,4-diamino-6-phenyl-1,3,5-triazine, 3,5-diamino-1,2,4-triazole, malonamide, arginine, piperidine, 2,5-piperazinedione, piperazine, piperazin-2-one-HCl, 1-(2-pyrimidyl)-piperazine dihydrochloride.

### Structure of the Compounds

The present invention is concerned with toxicity inhibiting compounds having various structures. Regardless of the specific structure of the present compound, the atoms of the compounds must be positioned in a way that allows for the compound to exert its inhibitory effect. Such inhibitory effect may result from the antagonistic binding of the compound to a receptor involved in cell toxicity and/or result from the binding of the compound to a therapeutic agent resulting in the prevention of a binding between the therapeutic agent and its receptor. By “binding” is meant a binding between the therapeutic agent and its corresponding receptor resulting in a cell toxic response.

### Charge

The compounds according to the invention are capable of accepting at least one proton, more preferably at least two protons because the structure of such compounds allows cell toxicity inhibition to be manifested. Accordingly, the compounds preferably have at least one amino group capable of functioning as a proton acceptor. More preferably, the compounds have two amino groups capable of functioning as a proton acceptor.

### Polybasic Charge Distribution

According to the invention it has been found that compounds comprising a polybasic charge distribution are particularly useful as inhibitors of induced cell toxicity. Thus, as discussed above, in a preferred embodiment of the invention the present compound has at least 1, such as 2 positive charges in solution, such as at least 2 positive charges. Preferably, the compound has 2 positive charges, preferably positively charged nitrogens, under physiological conditions.

By selecting the positive charges within an interval of from 1 to 300, such as 1 to 20, e.g. 1-10, it is normally possible to block a sufficient number of binding sites on the receptor responsible for mediating induced toxicity, such as the receptor megalin and/or the receptor cubilin and/or a co-receptor of megalin and cubilin. This results in the inhibition of uptake of the therapeutic agent responsible for inducing cell toxicity. In one embodiment, it is preferred that the polybasic charge distribution has the same charge distribution as the cell toxicity inducing therapeutic agent.

In the case of the receptor cubilin and the receptor megalin, a plausible explanation why a polybasic charge distribution is preferred may be that the binding of the aminoglycoside gentamicin to the receptor(s) is not necessarily dependent on the native conformation of the receptor, since reduction of disulfide-bridges does not significantly interfere with ligand binding. Moreover, the addition of EDTA, which depletes the presence of calcium and affects receptor stability, does not abolish binding. This indicates that the interaction between receptor and gentamicin may depend on simple ionic interactions rather than the overall confirmation of the receptor.

### Distance Between N Atoms

It has been found that the distance between the N′ and N″ atoms of the compounds (see all the present formulas) plays a role in the effectiveness of the present inhibitor. Thus, in one aspect of the invention the distance between the N′ and N″ atoms of the compounds of the invention is between 2.0 and 9.0 Ångstrøm. The distance is preferably between 6.0 and 8.0 Å, more preferably between 6.8 and 7.5 Å.

In another preferred embodiment, N′ and N″ are separated by 4-10 atoms, such as 5-9 atoms, e.g. 6-9 atoms, such as 7-9 atoms.

In other preferred embodiments, N′ and N″ and separated by 5-8 atoms, such as 5-7 atoms, preferably 6 or 7 atoms. Preferably, these atoms are C and/or N atoms, most preferably C atoms. If N′ and N″ are separated by 6 atoms, there are seven consecutive bonds between N′ and N″.

For example, in a preferred embodiment, X is a six-membered ring and R₁ and R₂ are in 1,4 (para) position relative to one another, wherein R₁ and R₂ are such that one atom separates each of N′ and N″ from X. The seven consecutive bonds separating N′ and N″ are in that case respectively: a bond from N′ to R₁, a bond from R₁ to the ring, three bonds in the ring, a bond from the ring to R₂ and a bond from R₂ to N″. Preferably R₁ and R₂ are carbons, optionally, but preferably not, substituted.

Furthermore, it is, in some embodiments, preferred that X constitutes a rigid, preferably flat ring structure.

In highly preferred embodiment, the compound has, under physiological conditions, at least two positively charged nitrogens that are separated by 4-10 atoms, such as 5-9 atoms, e.g. 6-9 atoms, such as 7-9 atoms. In other preferred embodiments, the compound has, under physiological conditions, at least two positively charged nitrogens that are separated by 5-8 atoms, such as 5-7 atoms, preferably 6 or 7 atoms. Preferably, these atoms are C and/or N atoms, most preferably C atoms. Physiological conditions wherein used herein, means conditions that are similar to those that can be found in the human body. Preferably, the pH in these conditions is between 6 and 7.5, such as between 6 and 7.

### Highly Preferred Specific Compounds

In a highly embodiment of the use of the invention, the compound is selected from the group consisting of: RC027, RC039, RC041, RC042, RC043, RC044, RC046, RC047, RC048, RC049, RC050, RC052, RC053, RC054, RC055, RC058, RC060, RC061, RC062, RC064, RC065, and RC083. The structures and names of these compounds are given in FIG. 1. An even more preferred group is the group consisting of: RC039, RC041, RC042, RC043, RC044, RC048, RC049, RC050, RC052, RC053, RC054, RC060, RC061, RC062, and RC065.

In another highly preferred embodiment, the compound is selected from the group consisting of RC075, RC076, RC077, RC078, RC080, RC081, and RC082, most preferably RC075.

In a yet other highly preferred embodiment, the compound is selected from the group consisting of RC051 and RC059.

In an even further highly preferred embodiment, the compound is selected from the group consisting of RC045, RC063, RC066, RC067, RC068, RC069, RC070, RC071, RC072, RC073, RC074 and RC079. An even more preferred group is the group consisting of: RC063, RC067, RC070, RC073, and RC079.

In another highly preferred embodiment, the compound is selected from the group consisting of RC084, RC085, and RC086.

In another preferred embodiment of the use of the invention, the compound is selected from the group consisting of trans 1,4-diaminocyclohexane, 1,3-cyclohexane bis(methylamine), 1,4-cyclohexane bis(methylamine), p-xylylene diamine, m-xylylene diamine, 1-(4-(-pyridyl)-piperazine, 2,5-dimethyl-1,4-xylylene-diamine dihydrochloride, α,α′-(dimethylamino)-p-xylene dihydrobromide, and compound RC051 (B13) shown in FIG. 1 (Sigma/Aldrich S111333).

In a more preferred embodiment, the compound is selected from the group consisting of trans 1,4-diaminocyclohexane, 1,3-cyclohexane bis(methylamine), 1,4-cyclohexane bis(methylamine), p-xylylene diamine, m-xylylene diamine, 2,5-dimethyl-1,4-xylylenediamine,dihydrochloride, α,α′-(dimethylamino)-p-xylene dihydrobromide.

In a further more preferred embodiment, the compound is selected from the group consisting of trans 1,4-diaminocyclohexane, p-xylylene diamine, m-xylylene diamine, 1-(4-(-pyridyl)-piperazine, α,α′-(dimethylamino)-p-xylene dihydrobromide.

In a further more preferred embodiment, the compound is selected from the group consisting of p-xylylene diamine, 1-(4-(-pyridyl)-piperazine, α,α′-(dimethylamino)-p-xylene dihydrobromide.

In a further more preferred embodiment, the compound is selected from the group consisting of p-xylylene diamine and α,α′-(dimethylamino)-p-xylene dihydrobromide.

Binding to the Receptor Cubilin and/or Megalin

The compounds of the present invention are all capable of binding to the receptor megalin and/or the receptor cubilin and/or a co-receptor of megalin and cubilin. Additionally the compounds according to the invention are capable of binding to the therapeutic agent. In case of binding to the receptor(s) it is of importance that the binding is effective in respect of blocking the binding of the therapeutic agent to the receptor. The receptor megalin for example comprises 50-150 binding sites for the therapeutic agent gentamicin and it is important for the effectiveness of the use of this invention that the compound is capable of inhibiting an effective amount of these binding sites.

Without being bound by theory the advantage of the present compound's ability of binding to the receptor cubilin and/or receptor megalin is the finding that these receptors are involved in aminoglycoside induced cell toxicity in the kidneys and the ear. Using the present compounds has an inhibitory effect on cell toxicity, such as nephrotoxicity and ototoxicity.

The compounds according to the invention may e.g. bind the receptor megalin in order to inhibit endocytosis or the receptor cubilin in order to reduce its sequestering and thereby inhibiting or reducing endocytosis.

It is also within the scope of the invention that the compound may bind to a co-receptor of megalin and cubilin.

### Novel Polymers

The invention further presents novel compounds to be used to reduce induced cell toxicity.

Thus, the invention presents a compound having the general formula

(A_{q}-Y)ₚ (IV)

wherein
A is independently selected from formula (I), (II) or (III) as defined herein, and wherein
Y is a spacer,
q is an integer of 1-100,
p is an integer of 1-100.

According to the invention q may be an integer of 1-100, 2-150, 2-100, 2-50, 2-10 or 5-95, for example 10-90, such as 15-90, for example 20-85, such as 25-80, for example 30-75, such as 35-70, for example 40-65, such as 45-60, for example 50-55.

Further, p of the formula may be an integer of 1-100, 2-150, 2-100, 2-50, 2-10 or 5-95, for example 10-90, such as 15-90, for example 20-85, such as 25-80, for example 30-75, such as 35-70, for example 40-65, such as 45-60, for example 50-55.

In one embodiment, A is independently or in combination selected from formula (I) and/or formula (II) and/or formula (III).

It is envisioned that one or more monomer(s) of the compound of formula (I) may be linked by a spacer to one or more monomer(s) of the compound of formula (II) or to one or more monomer(s) of the compound of formula (III).

In another embodiment one or more monomer(s) of the compound of formula (I) may be linked by a spacer to one or more monomer(s) of the compound of formula (I). Likewise, the compounds of formula (II) and (III), respectively, may also be linked to one or more monomers of the compounds of formula (II) and (III), respectively.

It is to be understood that the individual monomers of the above embodiments belonging to the same group formula (i.e. formula (I), (II), (III), respectively) are linked by spacers just like the individual monomers/polymers belonging to different group formulas are linked by spacers.

The combinations of the present compounds may be illustrated by the following examples:

formula (I)-formula (I)-spacer-formula (I)-formula (I)
formula (I)-spacer-formula (I)-formula (I)-formula (I)
formula (I)-spacer-formula (I)-spacer-formula (I)-formula (I)
formula (I)-formula (II)-spacer-formula (I)-formula (I)
formula (I)-spacer-formula (I)-formula (II)-formula (III)
formula (I)-spacer-formula (I)-spacer-formula (I)-formula (I)

The above combinations are merely non-limiting illustrations of the possible combinations possible within the scope of the present invention.

### Spacer

According to one embodiment of the invention the spacer is a covalent bond. In another preferred embodiment the spacer consists of from 2-12 atoms, such as C-atoms, such as from 4-10 atoms, such as C-atoms, preferably from 6-8 atoms, such as C-atoms.

The invention furthermore relates to the use of the above polymer compounds for the prophylaxis and/or treatment of induced cell toxicity as defined herein.

### Salts

A pharmaceutically acceptable salt may be any salt of the compounds mentioned. In particular, it means a pharmaceutically acceptable acid addition salt. Pharmaceutically acceptable acid addition salts of the compounds include salts derived from nontoxic inorganic acids such as hydrochloric, nitric, phosphoric, sulphuric, hydrobromic, hydriodic, hydrofluoric, phosphorous and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like.

### Pharmaceutical Composition

In a further aspect, the invention relates to a pharmaceutical composition comprising a compound as defined herein or a compound for which a use is described herein and pharmaceutically acceptable carriers, excipients or diluents therefore.

### Therapeutic Agent

The therapeutic agent according to the invention may be any therapeutic agent capable of causing organ damages due to intracellular accumulation in cells in the organs. In particular, the therapeutic agent is capable of accumulating in cells in the kidneys and/or inner ear, thus causing kidney damages as well as damages to the inner ear.

Thus in a further embodiment, the induced cell toxicity is a side-effect of a therapeutic agent, wherein the therapeutic agent is selected from acebutolol, acetazolamide, acyclovir, adefovir, albumin, alclofenac, alendronate, alitretinoin, altretamine, amikacin, amiloride, aminoglutethimide, amiodarone, amoxicillin, amoxicillin/clavulanic acid, amphotericin b, amphotericin b cholesteryl sulfate complex, amphotericin b lipid complex, amphotericin b liposome, amtolmetin, aniracetam, antacids, antazoline, anthraquinone laxatives, aprotinin, arbekacin, arginine, arsenic trioxide, asparaginase, aspirin, atenolol, atovaquone, auranofin, aurothioglucose, azacitidine, azathioprine, azlocillin, aztreonam, bacampicillin, bacitracin, bemetizide, benoxaprofen, betaine, bezafibrate, bismuth subcitrate, bleomycin, boric acid, brivudine, broxuridine, bumetanide, calcifediol, calcitriol, candesartan, candesartan/hydrochlorothiazide, canrenoate, capreomycin, captopril, carbenicillin, carboplatin, carmustine, carprofen, cefaclor, cefetamet, cefixime, cefmetazole, cefonicid, cefoperazone, cefoperazone/sulbactam, cefotaxime, cefotetan, cefoxitin, cefpirome, cefsulodin, ceftazidime, ceftezole, ceftibuten, ceftizoxime, ceftriaxone, cefuroxime, celecoxib, cephalexin, cephaloridine, cephalothin, cephapirin, cephradine, chlortetracycline, cidofovir, cilazapril, cimetidine, ciprofibrate, cisapride, cisplatin, clarithromycin, clodronate, clofibrate, cloxacillin, cocaine, codeine, colistin, corticotropin, cosyntropin, cotrimazine, cotrimoxazole, crisnatol, cyclacillin, cyclosporine, cysteamine, decitabine, delapril, delavirdine, demeclocycline, denileukin diftitox, desflurane, dextran, diatrizoate, diazoxide, dibekacin, diclofenac, diclofenac/misoprostol, dicloxacillin, dicumarol, didanosine, dihydroergotamine, dihydroergotamine/heparin, dihydrotachysterol, dirithromycin, dopamine, doxepin, doxorubicin hydrochloride liposome, doxycycline, edetate calcium disodium, edetate disodium, emetine, enflurane, enlimomab, epinephrine, epirubicin, ergocalciferol, ergotamine, erythromycin/sulfisoxazole, erythropoietins, ethanolamine oleate, ethyl chloride, etidronate, etodolac, etomidate, etretinate, everninomycin, fadrozole, fenbufen, fenofibrate, fenoprofen, fenoterol/ipratropium, flecainide, fleroxacin, floxacillin, flupirtine, flurbiprofen, formestane, foscarnet, fosinopril, fotemustine, framycetin, furosemide, gabexate, gadopentetate dimeglumine, gallium nitrate, gemcitabine, gemfibrozil, gentamicin, glycerin, gold sodium thiomalate, guanadrel, guanethidine, guar gum, halothane, hemiacidrin, hemin, hetastarch, homoharringtonine, hyaluronidase, hydrochlorothiazide, idarubicin, ifosfamide, imatinib, imipramine, indapamide, influenza vaccine, interferon alfa-2a, interferon alfa-2b, interferon beta, natural, interferon beta-1b, interferon gamma, interleukin-3, interleukin-4, interleukin-6, iobenguane 1-131, iodixanol, iohexol, iopamidol, iopanoic acid, iopentol, iopromide, iotrolan, ioversol, ioxaglate, ioxilan, ioxithalamate, irinotecan, irofulven, isepamicin, isoflurane, isoniazid, isoxicam, kanamycin, ketamine, ketoconazole, ketoprofen, ketorolac, lenograstim, levofloxacin, lincomycin, liposomal nystatin, lisinopril, lithium, lobaplatin, lomustine, lonidamine, lornoxicam, losartan, loxapine, lymphocyte immune globulin, mannitol, mebendazole, mefenamic acid, meglumine antimoniate, melarsoprol, meropenem, mesna, metaraminol, methacycline, methicillin, methimazole, methocarbamol, methotrexate, methoxamine, metrizamide, metronidazole, mezlocillin, milrinone, miltefosine, minocycline, minoxidil, mitoguazone, mitolactol, mitomycin, mitotane, molindone, morniflumate, morphine, moxalactam, muromonab-CD3, nabumetone, nafcillin, naproxen, nedaplatin, neomycin, netilmicin, niclosamide, nifedipine, niflumic acid, nifurtimox, nisoldipine, nitroprusside, norepinephrine, norfloxacin, ofloxacin, olsalazine, oxaliplatin, oxandrolone, oxaprozin, oxolinic acid, oxytetracycline, paclitaxel, pamidronate, paramethadione, paromomycin, pefloxacin, pemetrexed, pemirolast, penicillin G, pentamidine, pentostatin, peplomycin, perindopril, phenazopyridine, phenindione, phenobarbital, phenylbutazone, phenylpropanolamine, phenyloin, phosphates, piperacillin, pirarubicin, piretanide, piroxicam, plicamycin, poloxamer-188, polymyxin B, potassium perchlorate, praziquantel, proglumetacin, propylthiouracil, pyrimethamine/sulfadoxine, quinagolide, quinapril, quinine, rabbit antithymocyte globulin, raltitrexed, ranitidine, ranpirnase, recombinant human hemoglobin, rifampin, ritodrine, ritonavir, rofecoxib, rolitetracycline, rufloxacin, salsalate, sevoflurane, silver nitrate, silver sulfadiazine, simvastatin, sodium cellulose phosphate, sodium chloride, sodium fluoride, sodium stibogluconate, spironolactone, streptokinase, streptomycin, streptozocin, sulfamethoxazole, sulfasalazine, sulfinpyrazone, sulfisoxazole, sulindac, sulprostone, sultamicillin, suprofen, tacrolimus, tasonermin, teicoplanin, temafloxacin, teniposide, tenoxicam, tetracycline, thiopental, tiaprofenic acid, ticarcillin, ticrynafen, tiludronate, tiopronin, tobramycin, tocainide, tolazoline, tolmetin, torsemide, tramadol, triamterene, trimethadione, trimethaphan, trimethoprim, trimetrexate, trimipramine, troglitazone, tromethamine, typhoid vaccine, valsartan, vancomycin, zolimomab aritox, zomepirac, zopiclone, antisense RNA, PNA, siRNA or derivatives thereof.

In a preferred embodiment, the induced cell toxicity is a side-effect of a therapeutic agent, wherein the therapeutic agent is selected from the group consisting of aminoglycosides, such as arbekacin, gentamicin, kanamycin, neomycin, paramycin, ribostamycin, lividomycin, amikacin, dibekacin, butakacin, tobramycin, streptomycin, dihydrostreptomycin, sisomicin, verdamicin, netilmicin, and butikacin; cisplatin, amphotericin B, ifosfamide, polymyxin B, cyclophosphomide, methotrexate, aprotinin, ciclosporin, and valproate as well as therapeutic antibodies.

In a more preferred embodiment, the therapeutic agent is an aminoglycoside, such as gentamicin, arbekacin or kanamycin.

Also fusion proteins or fusion products used for medical treatment wherein one of the proteins is capable of binding the megalin or the receptor cubilin and/or a co-receptor of megalin and cubilin and the other protein/product causes cell toxicity when accumulating in the cells, may be used. In particular fusion products, wherein one part of the product is an antibody or IgG light chain, both capable of unspecifically binding to cubilin, and the other part of the product is cytotoxic, such as cancer treatment, may be co-administered with a compound according to the present invention in order to reduce organ damage, in particular kidney damage.

### Dosages

The dosage of the compound according to the invention depends on the compound in question; however, the amount of the compound is also closely related to the therapeutic agent co-administered with the compound as well as the dosage of said therapeutic agent.

For all methods of use disclosed herein for the compounds, the daily oral dosage regimen will preferably be from about 0.01 to about 500 mg/kg of total body weight, preferably 0.01-350 mg/kg, more preferably 0.1-200 mg/kg, such as 0.1-100 mg/kg of total body weight. The daily parenteral dosage regimen will be from about 0.001 to about 500 mg/kg of total body weight, preferably 0.01-350 mg/kg, more preferably 0.1-200 mg/kg, such as 0.1-100 mg/kg of total body weight.

The term “unit dosage form” as used herein refers to physically discrete units suitable as unitary dosages for human and animal individuals, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host. The dose administered should be an “effective amount” or an amount necessary to achieve an “effective level” in the individual patient.

Since the “effective level” is used as the preferred endpoint for dosing, the actual dose and schedule can vary, depending on inter-individual differences in pharmacokinetics, drug-distribution, and metabolism. The “effective level” can be defined, for example, as the blood or tissue level desired in the individual that corresponds to a concentration of one or more compounds according to the invention. Also, the effective level is depending on the therapeutic agent in question, and in particular on the concentration of the effective level in question.

Accordingly, in a preferred embodiment the ratio of the compound administered to the therapeutic agent administered is in the interval of from 200:1 mol:mol to 1:200 mol:mol, such as in the interval of from 100:1 mol:mol to 1:50 mol:mol, such as in the interval of from 50:1 mol:mol to 1:25 mol:mol

The compound may be administered in any suitable dosage regime, but is preferably administered with the same intervals as the therapeutic agent, preferably either shortly before or during administration of the therapeutic agent.

Most of the therapeutic agents according to this invention are administered parenterally, often intravenously. The compound according to the invention may be administered in any suitable manner according to the formulation thereof, it is however often preferred that the compound is administered parenterally, such as intravenously as the therapeutic agent.

### Medicament

According to the invention the present medicament is capable of binding to the receptor megalin. In a further embodiment the medicament is capable of binding to the receptor cubilin. In yet a further embodiment the medicament is capable of binding to a co-receptor of megalin and cubilin.

In another embodiment the medicament is capable of binding to a therapeutic agent capable of binding to the receptor megalin and/or the receptor cubilin and/or a co-receptor of megalin and cubilin.

### Combination Medicaments

The present invention further relates to a combination medicament comprising the compound according to the invention in combination with a therapeutic agent. Thus, the invention further relates to a combination medicament comprising a compound as disclosed by the invention or a compound for which a use is described herein and a therapeutic agent for simultaneous, separate or sequential use in therapy, preferably antimicrobial therapy. Preferably, the therapeutic agent is any one of the above-mentioned agents.

### Administration

When using the present medicament the compound and the therapeutic agent may be administered simultaneously, either as separate formulations or combined in a unit dosage form, or administered sequentially. The combination medicament may be formulated by co-formulating the compound according to, the invention with the therapeutic agent for simultaneous administration. In another embodiment the combination medicament is formulated as two separate medicaments for either simultaneous or sequential administration.

By the term “separate administration” is meant an initial administration of a first compound/medicament followed by secondary administration of a compound/medicament. The order of administration of the compounds/medicaments is not significant, and the time interval with which the first administration is followed by the second administration is not determined.

The main routes of drug delivery according to the present invention are intravenous, oral, and topical, as will be described below. Other drug administration methods, such as subcutaneous injection, which are effective to deliver the drug to a target site or to introduce the drug into the bloodstream, are also contemplated.

The mucosal membrane to which the pharmaceutical preparation of the invention is administered may be any mucosal membrane of the mammal to which the biologically active compound is to be given, e.g. in the nose, vagina, eye, mouth, genital tract, lungs, gastrointestinal tract, or rectum.

Compounds of the invention may be administered parenterally, that is by intravenous, intramuscular, subcutaneous, intranasal, intrarectal, intravaginal or intraperitoneal administration. The subcutaneous and intramuscular forms of parenteral administration are generally preferred. Appropriate dosage forms for such administration may be prepared by conventional techniques. The compounds may also be administered by inhalation, such as by intranasal and oral inhalation administration.

The compounds according to the invention may be administered with at least one other compound. The compounds may be administered simultaneously, either as separate formulations or combined in a unit dosage form, or administered sequentially. The combination medicament may be formulated by co-formulating the compound according to the invention with the therapeutic agent for simultaneous administration. In another embodiment the combination medicament is formulated as two separate medicaments for either simultaneous or sequential administration.

Pharmaceutical compositions containing a compound of the present invention may be prepared by conventional techniques, e.g. as described in Remington: The Science and Practice of Pharmacy 1995, edited by E. W. Martin, Mack Publishing Company, 19th edition, Easton, Pa. The compositions may appear in conventional forms, for example capsules, tablets, aerosols, solutions, suspensions or topical applications.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilising and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The parenteral formulations will typically contain from about 0.5 to about 25% by weight of the active ingredient in solution. Preservatives and buffers may be used. In order to minimise or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

### EXAMPLES

The following are non-limiting examples illustrating the invention.

### Example 1

### In Vitro Test

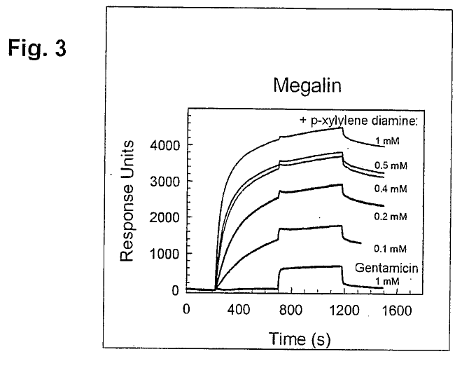

Inhibition of gentamicin binding to immobilized megalin by p-xylylene diamine was assessed by surface plasmon resonance (SPR) analysis on a Biacore 2000 instrument (Biacore, Uppsala, Sweden). Megalin purified from rabbit kidneys as described in Birn et al. (J. Biol. Chem., 1997, Vol. 272, No. 42, 26497-26504) was immobilized in a concentration of 28-40 fmol/mm². Samples were dissolved in 10 mM Hepes, 150 mM NaCl, 1.5 mM CaCl₂, 1 mM EGTA, 0.005% Tween-20 pH 7.4. The same buffer was used as running buffer. At time zero, p-xylylene diamine at the concentrations indicated (FIG. 3) were applied to the chip and binding as expressed in relative response units (RU), i.e. the difference in response between megalin and a control flow channel, was measured. After 700 sec., 1 mM gentamicin was co-injected with buffer alone or various concentrations of p-xylylene diamine. Inhibition of gentamicin binding is scored as the difference in RU units obtained by injection of gentamicin alone and in the presence of the antagonist. Regeneration of the sensor chip after each analysis cycle was performed with 1.6 M glycine-HCl buffer pH 3.0.

FIG. 3 shows that increasing concentrations of p-xylylene diamine (RC043/B5) inhibit binding of gentamicin. 50% inhibition is obtained at an antagonist concentration of about 0.1 mM.

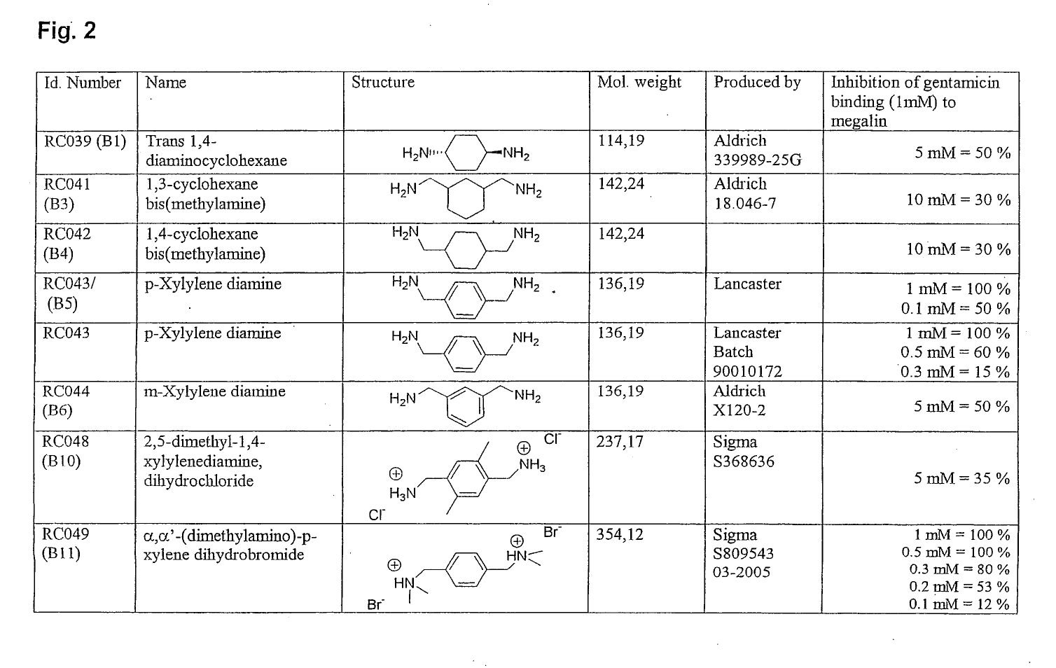

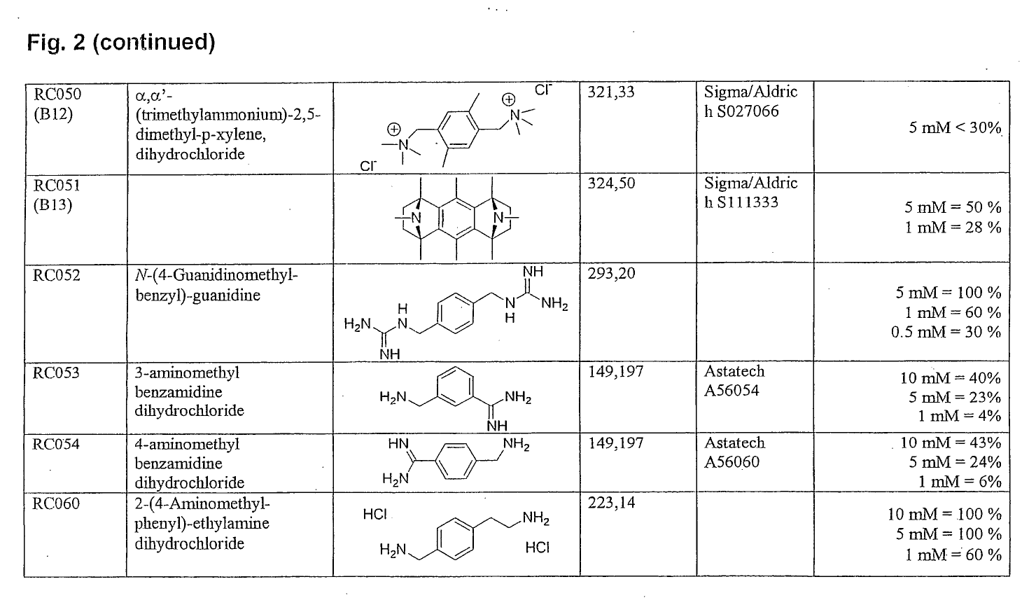

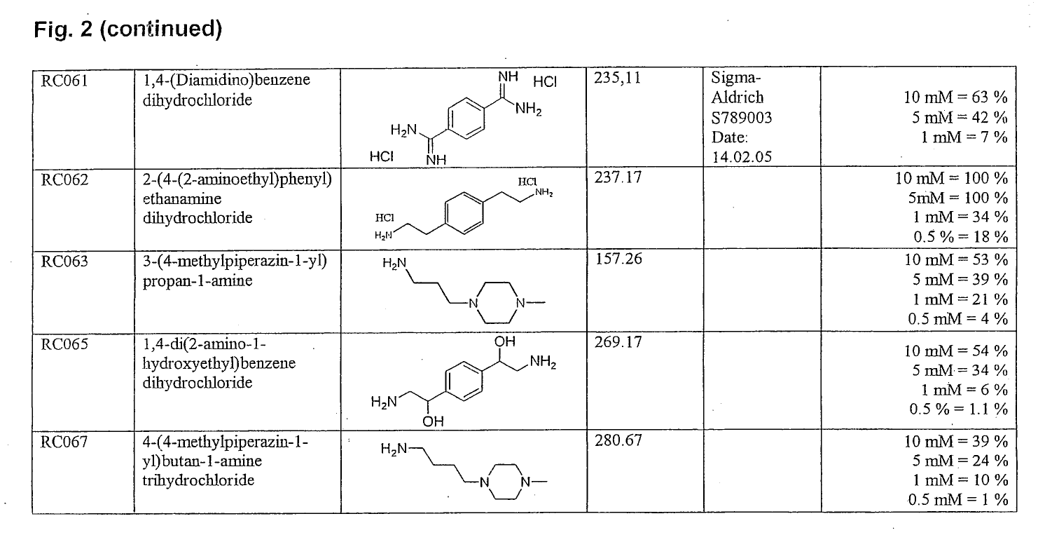

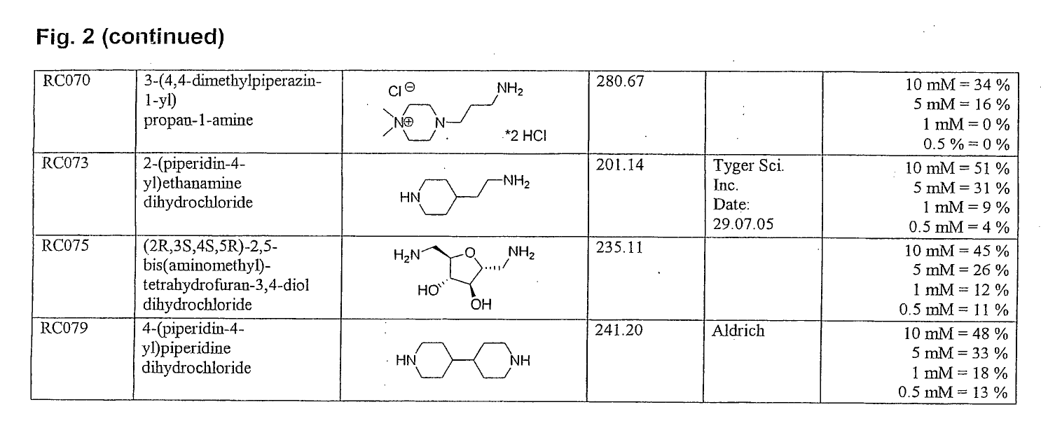

In similar experiments, a number of the compounds shown in FIG. 1 was tested. The results are given in FIG. 2. The exact concentration of RC048 (B10) in the experiment was uncertain, as the compound was not entirely dissolved.

### Example 2

The effect of p-xylylene diamine (RC043/B5) was tested in vivo. Gentamicin uptake in mouse kidneys following intraperitoneal (i.p.) administration of the compound was measured. 50 micrograms per kg of tritiated gentamicin was injected, with different concentrations of the inhibitor per mouse. This equals 1% of the clinical dose of gentamicin used in patients.

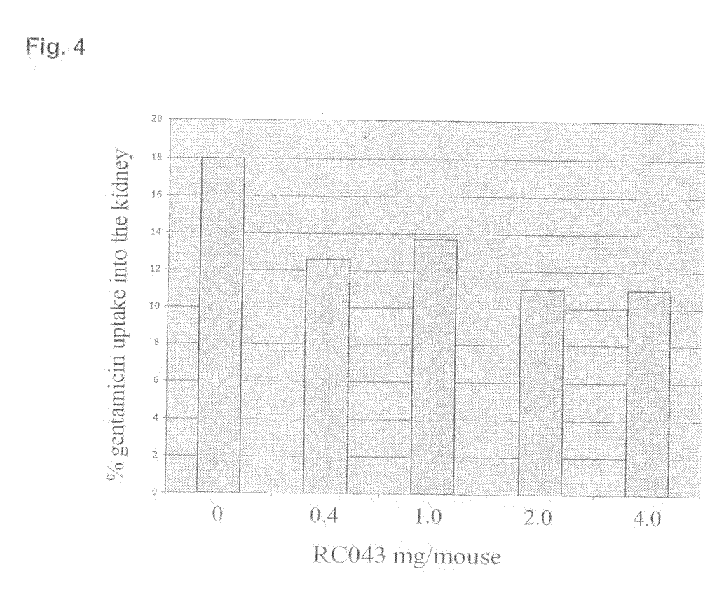

Results are shown in FIG. 4.

1. A method for preventing or treating nephrotoxicity comprising administering an effective amount of a medicament comprising a compound comprising a structure of the general formula (I)
R₇-R₁-X-R₈
wherein 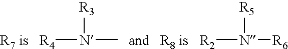
wherein
X is a bond or an aromatic, a carbocyclic, a heterocyclic or a heteroaromatic structure having 1-3 rings, 3-8 ring members in each and 0 to 3 heteroatoms, wherein each ring may be substituted at least once, wherein the substituents are selected from: O, OH, phenyl, halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, nitro, Oalkyl, Oacyl, aminoalkyl and aminodialkyl,
R1 is a bond, or C1-10 alkyl, optionally substituted at least once, wherein the substituents are selected from: O, OH, phenyl, amine (NH2), imine (NH), halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, or nitro, wherein any of the carbons in said C1-10 alkyl is optionally replaced by oxygen, nitrogen, sulphur, or silicon,
R2 is a bond or C1-10 alkyl, optionally substituted at least once, wherein the substituents are selected from: O, OH, phenyl, amine (NH2), imine (NH), halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, or nitro, wherein any of the carbons in said C1-10 alkyl is optionally replaced by oxygen, nitrogen, sulphur, or silicon,
R3, R4, R5, and R6 individually are selected from: a bond connecting to X of formula (I), hydrogen, OH, trifluoromethyl, cyano, amino, nitro, alkyl, alkenyl, alkynyl, phenyl, benzyl, halogen, substituted lower alkyl, aryl, heterocycloalkyl, heteroaryl, aryl-(C1-4)-alkyl, heteroaryl-(C1-4)-alkyl, heterocyclyl-(C1-4)-alkyl, cycloalkylalkyl, cycloalkyl,
optionally further substituted one or more times with C, S, N, O, OH, phenyl, amine (NH), halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, or nitro,
wherein one or more of R3, R4, R5, and R6 is optionally linked to another substituent R1-R8 and/or to X, thereby forming a ring,
wherein N′ and N″ are separated by at least 4 atoms,
wherein N′ and N″ optionally have a further group attached thus forming a quaternary ammonium,
with the proviso that the compound is not a diaminoalkyl, wherein both the alkyl group and the amino groups have no substitutions,
and the proviso that the compound is not 3-methylamino-1-(4-methylpiperazino)-2-propanole, 4-piperazinoaniline, 2-[4-(2-aminoethyl)piperazin-1-yl]ethylamine, arginine, or 1-(2-pyrimidyl)-piperazine dihydrochloride
or a pharmaceutically acceptable addition salt or hydrate of said compound.

2. The method according to claim 1, wherein X is an aromatic, a carbocyclic, a heterocyclic or a heteroaromatic structure having 1-3 rings, 3-8 ring members and 0 to 3 heteroatoms in each ring, wherein each ring may be substituted at least once, wherein the substituents are selected from O, OH, phenyl, halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, nitro, Oalkyl, Oacyl, aminoalkyl and aminodialkyl.

3. The method according to claim 1, wherein X is an aromatic, a carbocyclic, a heterocyclic or a heteroaromatic structure having 1-3 rings, 5-6 ring members and 1 or 2 heteroatoms in each ring, wherein each ring may be substituted at least once, wherein the substituents are selected from O, OH, phenyl, halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, nitro, Oalkyl, Oacyl, aminoalkyl and aminodialkyl.

4. The method according to claim 1, wherein X is an aromatic or a carbocyclic structure having 1-3 rings, 5-6 ring members and no heteroatoms in each ring, wherein each ring may be substituted at least once, wherein the substituents are selected from O, OH, phenyl, halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, nitro, Oalkyl, Oacyl, aminoalkyl and aminodialkyl.

5. The method according to claim 2, wherein X is an aromatic, a carbocyclic, a heterocyclic or a heteroaromatic structure having 1-2 rings, 3-8 ring members in each and having 0 to 3 heteroatoms, wherein each ring optionally is substituted.

6. The method according to claim 2, wherein X is an aromatic, a carbocyclic, a heterocyclic or a heteroaromatic structure having 1 ring with 3-8 ring members and having 0 to 3 heteroatoms, said ring optionally being substituted.

7. The method according to claim 2, wherein X is an aromatic, a carbocyclic, a heterocyclic or a heteroaromatic structure having 1 ring with 4-7 ring members and having 0 to 3 heteroatoms, said ring optionally being substituted.

8. The method according to claim 2, wherein X is an aromatic, a carbocyclic, a heterocyclic or a heteroaromatic structure having 1 ring with 5-6 ring members and having 0 to 3 heteroatoms, said ring optionally being substituted.

9. The method according to claim 2, wherein X is an aromatic, a carbocyclic, a heterocyclic or a heteroaromatic structure having 1 ring with 5 ring members and having 0 to 1 heteroatoms, said ring optionally being substituted.

10. The method according to claim 2, wherein X is selected from the group consisting of cyclopentyl, furan, thiophene, pyrrole, imidazole, oxazole, and pyrrolidene, preferably the group consisting of furan and pyrrole.

11. The method according to claim 2, wherein X is selected from the group consisting of pyridine, pyrimidine, cyclohexyl, and phenyl, preferably cyclohexyl and phenyl.

12. The method according to claim 2, wherein X is an aromatic, a heteroaromatic or a 5 or 6 membered saturated ring containing 0-2 oxygen atoms, optionally substituted at least once, wherein the substituents are selected from O, OH, phenyl, halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, nitro, Oalkyl, Oacyl, aminoalkyl and aminodialkyl,
R1 is C1-10 alkyl, optionally substituted at least once, wherein the substituents are selected from O, OH, phenyl, amine (NH2), imine (NH), halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, and nitro, wherein any of the carbons in said C1-10 alkyl is optionally replaced by oxygen, nitrogen, sulphur, or silicon,
R2 is C1-10 alkyl, optionally substituted at least once, wherein the substituents are selected from O, OH, phenyl, amine (NH2), imine (NH), halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, and nitro, wherein any of the carbons in said C1-10 alkyl is optionally replaced by oxygen, nitrogen, sulphur, or silicon,
R3, R4, R5, and R6 individually are selected from: a bond connecting to X of formula (I), hydrogen, OH, trifluoromethyl, cyano, amino, nitro, alkyl, alkenyl, alkynyl, phenyl, benzyl, halogen, substituted lower alkyl, aryl, heterocycloalkyl, heteroaryl, aryl-(C1-4)-alkyl, heteroaryl-(C1-4)-alkyl, heterocyclyl-(C1-4)-alkyl, cycloalkylalkyl, cycloalkyl, optionally further substituted one or more times with C, S, N, O, OH, phenyl, amine (NH), halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, or nitro.

13. The method according to claim 2, wherein X is an aromatic, carbocyclic, a heterocyclic or a heteroaromatic structure having 1 ring with 6 ring members and having 0 to 1 heteroatoms, said ring optionally being substituted.

14. The method according to claim 2, wherein X is a dicyclohexylmethane.

15. The method according to claim 2, wherein X comprises a heterocyclic ring comprising at least one oxygen atom.

16. The method according to claim 15, wherein the compound comprises a structure of the formula V: 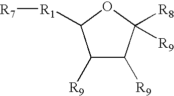
wherein each R9, independently is H, OH, Oalkyl, Oacyl, halogen, alkyl, amino, aminoalkyl, aminodialkyl or aryl.

17. The method according to claim 15, wherein the compound comprises a structure of the formula VI: 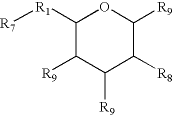
wherein each R9, independently is H, OH, Oalkyl, Oacyl, halogen, alkyl, amino, aminoalkyl, aminodialkyl or aryl.

18. The method according to claim 15, wherein the compound comprises a structure of the formula VII: 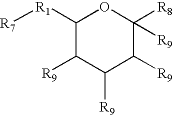
wherein each R9, independently is H, OH, Oalkyl, Oacyl, halogen, alkyl, amino, aminoalkyl, aminodialkyl or aryl.

19. The method according to claim 15, wherein at least one of the R9 groups is H and at least one of the R9 groups is OH.

20. The method according to claim 15, wherein R7 comprises a guanidine group.

21. The method according to claim 15, wherein R8 comprises a guanidine group.

22. The method according to claim 1, wherein R1 is a bond, or C1-10 alkyl, optionally substituted at least once, wherein the substituents are selected from O, OH, phenyl, amine (NH2), imine (NH), halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, and nitro.

23. The method according to claim 1, wherein R1 is a bond, or C1-4 alkyl, optionally substituted at least once, wherein the substituents are selected from O, OH, phenyl, amine (NH2), imine (NH), halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, and nitro.

24. The method according to claim 1, wherein R1 is a bond, or C1-2 alkyl, optionally substituted at least once, wherein the substituents are selected from O, OH, phenyl, amine (NH2), imine (NH), halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, and nitro.

25. The method according to claim 1, wherein R1 is a C1 or C2 alkyl, said alkyl being optionally substituted at least once, wherein the substituent(s) is selected from O, OH, phenyl, amine (NH2), imine (NH), halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, and nitro.

26. The use method according to claim 25, wherein R1 is a C1 or C2 alkyl, substituted at least once with an imine group or substituted at least once with an OH group.

27. The method according to claim 1, wherein R1 is C1.

28. The method according to claim 1, wherein R1 is a bond.

29. The method according to claim 1, wherein R2 is a bond or C1-10 alkyl, optionally substituted at least once, wherein the substituents are selected from O, OH, phenyl, amine (NH2), imine (NH), halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, and nitro.

30. The method according to claim 1, wherein R2 is a bond or C1-4 alkyl, optionally substituted at least once, wherein the substituents are selected from O, OH, phenyl, amine (NH2), imine (NH), halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, and nitro.

31. The method according to claim 1, wherein R2 is a bond or C1-2 alkyl, optionally substituted at least once, wherein the substituents are selected from O, OH, phenyl, amine (NH2), imine (NH), halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, and nitro.

32. The method according to claim 1, wherein R2 is a C1 or C2 alkyl, said alkyl being optionally substituted at least once, wherein the substituents are selected from O, OH, phenyl, amine (NH2), imine (NH), halogen, alkyl, alkenyl or alkynyl, substituted lower alkyl, substituted lower alkenyl or alkynyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, trifluoromethyl, cyano, amino, and nitro.

33. The method according to claim 32, wherein R2 is a C1 or C2 alkyl, substituted at least once with an imine group or substituted at least once with an OH group.

34. The method according to claim 1, wherein R2 is C1.

35. The method according to claim 1, wherein R2 is a bond.

36. The method according to claim 1, wherein R1 is a bond and R2 is a C1-2 alkyl.

37. The method according to claim 1, wherein at least one of R3, R4, R5 and R6 is hydrogen.

38. The method according to claim 1, wherein at least two of R3, R4, R5 and R6 are hydrogen.

39-48. (canceled)

49. The method according to claim 1, wherein X, R2, N″ and R5 together form a ring Y, said ring Y being a piperazine or piperidine ring, thereby forming the general formula VIII:

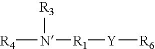

50-55. (canceled)

56. The method according to claim 1, wherein X is a bond.

57-60. (canceled)

61. The method according to claim 1, wherein said compound has a polybasic charge distribution.

62. The method according to claim 1, where the compound under physiological conditions has at least two positively charged nitrogens.

63-68. (canceled)

69. The method according to claim 1, wherein the compound is selected from the group consisting of: RC027, RC039, RC041, RC042, RC043, RC044, RC046, RC047, RC048, RC049, RC050, RC052, RC053, RC054, RC055, RC058, RC060, RC061, RC062, RC064, RC065, and RC083.

70. The method according to claim 1, wherein the compound is selected from the group consisting of RC075, RC076, RC077, RC078, RC080, RC081, and RC082.

71. The method according to claim 1, wherein the compound is selected from the group consisting of RC051 and RC059.

72. The method according to claim 1, wherein the compound is selected from the group consisting of RC045, RC063, RC066, RC067, RC068, RC069, RC070, RC071, RC072, RC073, RC074 and RC079.

73. The method according to claim 1, wherein the compound is selected from the group consisting of RC084, RC085, and RC086.

74. The method according to claim 1, wherein the compound is capable of binding to the receptor megalin and/or the receptor cubilin.

75. The method according to claim 1, wherein the nephrotoxicity is a side-effect of a therapeutic agent, wherein the therapeutic agent is selected from arbekacin, capreomycin, captopril, carbenicillin, carboplatin, carmustine, carprofen, cefaclor, cefetamet, cefixime, cefmetazole, cefonicid, cefoperazone, cefoperazone/sulbactam, cefotaxime, cefotetan, cefoxitin, cefpirome, cefsulodin, ceftazidime, ceftezole, ceftibuten, ceftizoxime, ceftriaxone, cefuroxime, celecoxib, cephalexin, cephaloridine, cephalothin, cephapirin, cephradine, chlortetracycline, cidofovir, cilazapril, cimetidine, ciprofibrate, cisapride, cisplatin, clarithromycin, clodronate, clofibrate, cloxacillin, cocaine, codeine, colistin, corticotropin, cosyntropin, cotrimazine, cotrimoxazole, crisnatol, cyclacillin, cyclosporine, cysteamine, decitabine, delapril, delavirdine, demeclocycline, denileukin diftitox, desflurane, dextran, diatrizoate, diazoxide, dibekacin, diclofenac, diclofenac/misoprostol, dicloxacillin, dicumarol, didanosine, dihydroergotamine, dihydroergotamine/heparin, dihydrotachysterol, dirithromycin, dopamine, doxepin, doxorubicin hydrochloride liposome, doxycycline, edetate calcium disodium, edetate disodium, emetine, enflurane, enlimomab, epinephrine, epirubicin, ergocalciferol, ergotamine, erythromycin/sulfisoxazole, erythropoietins, ethanolamine oleate, ethyl chloride, etidronate, etodolac, etomidate, etretinate, everninomycin, fadrozole, fenbufen, fenofibrate, fenoprofen, fenoterol/ipratropium, flecainide, fleroxacin, floxacillin, flupirtine, flurbiprofen, formestane, foscarnet, fosinopril, fotemustine, framycetin, furosemide, gabexate, gadopentetate dimeglumine, gallium nitrate, gemcitabine, gemfibrozil, gentamicin, glycerin, gold sodium thiomalate, guanadrel, guanethidine, guar gum, halothane, hemiacidrin, hemin, hetastarch, homoharringtonine, hyaluronidase, hydrochlorothiazide, idarubicin, ifosfamide, imatinib, imipramine, indapamide, influenza vaccine, interferon alfa-2a, interferon alfa-2b, interferon beta, natural, interferon beta-1b, interferon gamma, interleukin-3, interleukin-4, interleukin-6, iobenguane I-131, iodixanol, iohexol, iopamidol, iopanoic acid, iopentol, iopromide, iotrolan, ioversol, ioxaglate, ioxilan, ioxithalamate, irinotecan, irofulven, isepamicin, isoflurane, isoniazid, isoxicam, kanamycin, ketamine, ketoconazole, ketoprofen, ketorolac, lenograstim, levofloxacin, lincomycin, liposomal nystatin, lisinopril, lithium, lobaplatin, lomustine, lonidamine, lornoxicam, losartan, loxapine, lymphocyte immune globulin, mannitol, mebendazole, mefenamic acid, meglumine antimoniate, melarsoprol, meropenem, mesna, metaraminol, methacycline, methicillin, methimazole, methocarbamol, methotrexate, methoxamine, metrizamide, metronidazole, mezlocillin, milrinone, miltefosine, minocycline, minoxidil, mitoguazone, mitolactol, mitomycin, mitotane, molindone, morniflumate, morphine, moxalactam, muromonab-CD3, nabumetone, nafcillin, naproxen, nedaplatin, neomycin, netilmicin, niclosamide, nifedipine, niflumic acid, nifurtimox, nisoldipine, nitroprusside, norepinephrine, norfloxacin, ofloxacin, olsalazine, oxaliplatin, oxandrolone, oxaprozin, oxolinic acid, oxytetracycline, paclitaxel, pamidronate, paramethadione, paromomycin, pefloxacin, pemetrexed, pemirolast, penicillin G, pentamidine, pentostatin, peplomycin, perindopril, phenazopyridine, phenindione, phenobarbital, phenylbutazone, phenylpropanolamine, phenyloin, phosphates, piperacillin, pirarubicin, piretanide, piroxicam, plicamycin, poloxamer-188, polymyxin B, potassium perchlorate, praziquantel, proglumetacin, propylthiouracil, pyrimethamine/sulfadoxine, quinagolide, quinapril, quinine, rabbit antithymocyte globulin, raltitrexed, ranitidine, ranpirnase, recombinant human hemoglobin, rifampin, ritodrine, ritonavir, rofecoxib, rolitetracycline, rufloxacin, salsalate, sevoflurane, silver nitrate, silver sulfadiazine, simvastatin, sodium cellulose phosphate, sodium chloride, sodium fluoride, sodium stibogluconate, spironolactone, streptokinase, streptomycin, streptozocin, sulfamethoxazole, sulfasalazine, sulfinpyrazone, sulfisoxazole, sulindac, sulprostone, sultamicillin, suprofen, tacrolimus, tasonermin, teicoplanin, temafloxacin, teniposide, tenoxicam, tetracycline, thiopental, tiaprofenic acid, ticarcillin, ticrynafen, tiludronate, tiopronin, tobramycin, tocainide, tolazoline, tolmetin, torsemide, tramadol, triamterene, trimethadione, trimethaphan, trimethoprim, trimetrexate, trimipramine, troglitazone, tromethamine, typhoid vaccine, valsartan, vancomycin, zolimomab aritox, zomepirac, zopiclone, antisense RNA, PNA, siRNA or derivatives thereof.

76-77. (canceled)

78. A compound having the general formula of
(A_{q}-Y)ₚ (IV)
wherein
A is independently selected from formula (I), and
wherein
Y is a spacer,
q is an integer of 1-100,
p is an integer of 1-100.

79. The compound according to claim 0, wherein the spacer is a covalent bond.

80. The compound according to claim 0, wherein the spacer consists of from 2-12 atoms.

81. A method for preventing or treating nephrotoxicity comprising administering an effective amount of a medicament comprising a compound as defined in claim 78.

82. A combination medicament comprising a compound as defined in claim 1 and a therapeutic agent for simultaneous, separate or sequential use in therapy.

83. A pharmaceutical composition comprising a compound as defined in claim 1 and pharmaceutically acceptable carriers, excipients or diluents therefor.
